(19) United States
(12) Patent Application Publication
(10) Pub. No.: US 2017/0231535 A1
(43) Pub. Date: Aug. 17, 2017

(54) AUDIOMETER

(71) Applicant: AUDYX SYSTEMS LTD., Jerusalem (IL)

(72) Inventor: Yves Samuel LASRY, Nantes (FR)

(21) Appl. No.: 15/504,325

(22) PCT Filed: Nov. 9, 2014
(86) PCT No.: PCT/IB2014/065903

§ 371 Date: Feb. 16, 2017

Related U.S. Application Data
(60) Provisional application No. 62/038,305, filed on Aug. 17, 2014.

(51) Int. Cl. A61B 5/12; H04R 29/00; A61B 5/00

(52) U.S. Cl. CPC A61B 5/123; A61B 5/0022; A61B 5/6898; H04R 29/001; A61B 2560/0223; A61B 2560/045; H04R 1/1008

(57) ABSTRACT

An audiometer comprising: a database storing a non-transitory digital test signal associated with a desired test sound; a digital to analog converter (DAC) that converts the digital test signal to an analog signal; headphones that produce a sound responsive to the analog signal; a microphone that registers the sound; and a controller that adjusts the digital test signal so that the sound produced by the headphones has acceptable fidelity relative to the desired test sound to have the sound be useable in a hearing test.

### RELATED APPLICATIONS

This application is claims benefit under 35 U.S.C. 119(e) of U.S. Provisional Application 62/038,305 filed Aug. 17, 2014, the disclosure of which is incorporated herein by reference.

### TECHNICAL FIELD

Embodiments of the invention relate to audiometry equipment and methods.

### BACKGROUND

An audiometer is a system for performing audiometric tests to measure hearing acuity. The audiometer is configured to provide a set of sounds (“test sounds”) having well-defined parameters, which are presented to a subject as stimuli to which he or she is prompted to respond. The test sound may be produced by a speaker such as air-conduction over-ear headphones, earphone inserts or an open field speaker. The “speaker” may be a bone-conduction vibrator. The speaker may be referred to herein generically as “headphones”. By way of example, a “gold standard” headphone for use in audiometry is the TDH39 over-ear headphone. A test sound may, for example, comprise a pure tone characterized by a particular frequency and a particular intensity or may comprise a complex sound that is a combination of two or more pure tones. A complex sound may be characterized by a substantially continuous frequency spectrum. The complex sound may optionally be a natural sound or a vocal sound.

Audiometric tests may measure one or more of various parameters relating to hearing acuity, including by way of example threshold of audibility (an intensity of a sound to be just audible to a subject) at each of a plurality of frequencies in a range of frequencies, ability to distinguish between different sound intensities or different sound frequencies, and recognize speech or distinguish speech in the presence of background noise.

Responses of the subject to the test sound may be recorded and analyzed to produce an assessment of the subject's hearing acuity. The subject response may be the subject's acknowledgement that a test sound was perceived, the subject's indication that an audible sound is no longer heard, or a correct identification by the subject of the presented test sound. For example, the subject may be presented with a test sound that is a spoken word, and the subject may respond by correctly repeating the presented word or selecting the correct word from a selection provided to the subject on a computer screen.

Because audiometric tests generally require that test sounds be produced by the headphones at well-defined frequencies and intensities, an audiometer is typically calibrated regularly to ensure that an actual acoustic output that is generated by the headphones is substantially the same as a desired test sound. Typically, calibration of an audiometer requires a trained technician with specialized equipment to go to a testing site where the audiometer is located.

### SUMMARY

An aspect of an embodiment of the invention relates to providing an audiometer that is relatively easily calibrated remotely without requiring presence of a trained technician at the physical site of the audiometer and is relatively easily configured to perform any of various selectable audiometric tests on a subject. For convenience of presentation, the audiometer in accordance with an embodiment of the invention may be referred to herein as a “remote calibration audiometer” or “RCA”.

In accordance with an embodiment of the invention, the RCA comprises a computer system (“RCA computer system”) that comprises a database of digital signals (“digital test signals”), stored in a non-transitory computer readable medium, for controlling headphones to produce desired test sounds, and a calibrated microphone that registers the test sounds produced by the headphones. A test sound may be, by way of example, a tone, a warble, white noise, pink noise, or a vocal sound. Optionally, the RCA comprises the headphones. An aspect of an embodiment of the invention relates to providing an adapter that acoustically couples the headphones to the microphone for the purpose of determining fidelity of test sounds produced by the headphones.

The digital test signals may be converted into analog test signals for producing the test sound in the headphones through a digital-to-analog converter (DAC), which may be housed in the headphones, the computer system, or in a separate component that connects the headphones and the computer system. The sound registered by the microphone may be converted into a “digital calibration signal” through an audio-to-digital converter (ADC), which may be housed in the microphone, the computer system, or in a separate component that connects the microphone and the computer system.

The RCA computer system may comprise a controller to implement a calibration procedure. Optionally, the controller comprises a computer instruction set stored in a non-transitory computer readable medium that is executable to implement the calibration procedure. The calibration procedure may determine, optionally remotely, fidelity of test sounds produced by the headphones by comparing the test sound registered by the microphone with the desired test sound. The calibration procedure may calibrate the RCA in accordance with the determined fidelity. Fidelity of a test sound produced by the RCA refers to how accurately a test sound generated responsive to a given digital test signal by the headphones reproduces a test sound that the given digital test signal is desired to produce. Calibrating the RCA in accordance with an embodiment of the invention may refer to adjusting a given digital test signal so that the test sound responsive to the given digital test signal produced in the headphones has acceptable fidelity for use in audiometry test of a subject.

The RCA computer system may optionally be centralized within a single computer device, or be a “distributed system” with code and hardware components located in different locations. In an embodiment of the invention, the RCA computer system may comprise a local computer device located at an audiometry test site together with the headphones and/or a remote computer system that is located outside of the audiometry test site with which the local computer device communicates. The remote computer system may optionally be a remote server or a remote distributed system. The remote distributed system may be referred to herein as a “cloud computer system.”

In the discussion, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical features that appear in more than one figure are generally labeled with a same label in all the figures in which they appear. A label labeling an icon representing a given feature of an embodiment of the invention in a figure may be used to reference the given feature. Dimensions of features shown in the figures are chosen for convenience and clarity of presentation and are not necessarily shown to scale.

FIG. 1 shows a schematic illustration of a RCA in accordance to an embodiment of the invention;

FIGS. 2A-2C show schematic illustrations of a calibration process conducted with the RCA according to an embodiment of the invention; and

FIGS. 3A-3D show schematic illustrations of the headphone and microphone coupled with an adapter, in accordance with an embodiment of the invention.

### DETAILED DESCRIPTION

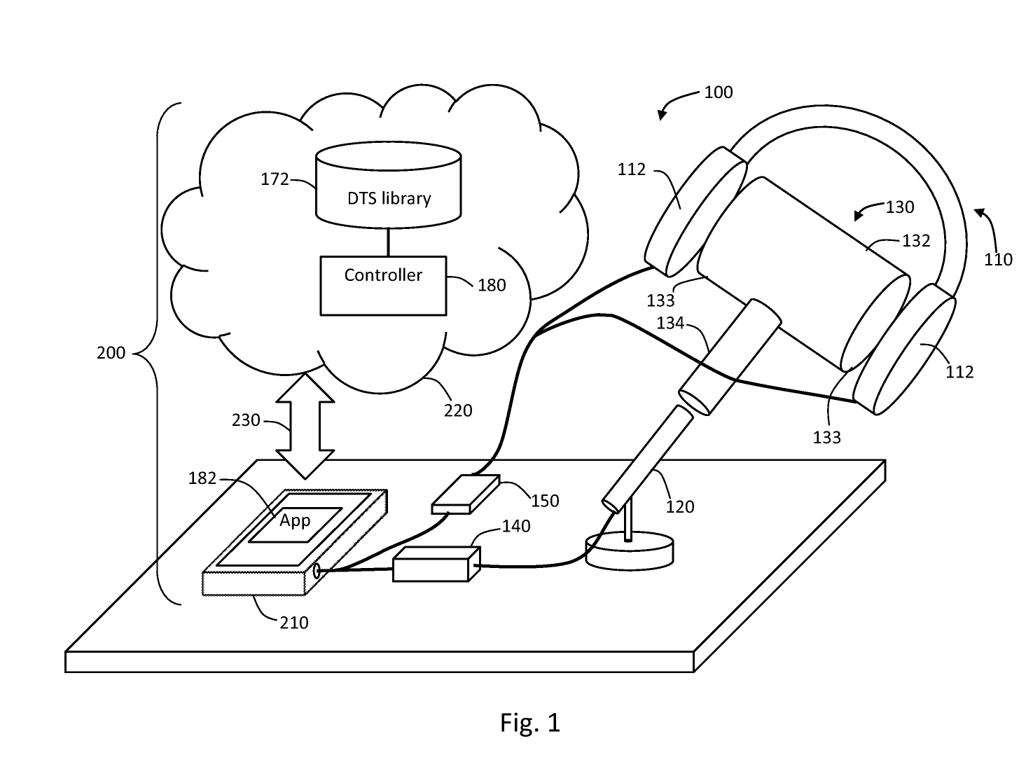

FIG. 1 schematically shows a RCA 100 in accordance to an embodiment of the invention. RCA 100 may comprise a calibrated microphone 120 and a RCA computer system 200. RCA computer system 200 may comprise a database (“DTS database”) 172 comprising a plurality of digital test signals, stored in non-transitory computer-readable media, that may be used to control headphones 110 having earpieces 112 to produce test sounds. Optionally, RCA 100 comprises headphones 110. The test sound may be, by way of example, a tone, a warble, white noise, pink noise, or a vocal sound. DTS database 172 may comprise an identification of a desired test sound corresponding to each of the digital test signals. RCA computer system 200 may comprise a controller 180 that controls a calibration procedure to determine the fidelity of a given test sound. If the fidelity of the registered test sound does not have acceptable fidelity, the controller may modify a digital test signal corresponding to the given test sound in accordance with the determined fidelity of the registered test sound so that the RCA subsequently reproduces the given test sound with acceptable fidelity.

Various standards of fidelity for reproduction of sound for use in audiometry are known in the art. For example, ISO (International Standards Organization) and ANSI (American National Standards Institute) define acceptable fidelity by giving a tolerance for different parameters. By way of example, ISO 60645-1 defines acceptable fidelity for intensity as being within +/−3.7 dB (decibel) relative to a desired intensity for a pure tone having a frequency between 125 Hz (Herz) and 4000 Hz, and as being within +/−6.8 dB relative to a desired intensity for a pure tone having a frequency between 4000 Hz and 8000 Hz. ISO 60645-1 defines acceptable fidelity for frequency of a pure tone as being within +/−2.5% or within 1.5% of a desired frequency.

Optionally, acceptable fidelity for a test sound produced by headphones of RCA 100 in accordance with an embodiment of the invention comprises having an intensity within a tolerance of about +/−7 dB or less relative to a desired test sound, having an intensity within a tolerance of about +/−4 dB or less relative to a desired test sound, having an intensity within a tolerance of about +/−3 dB or less relative to a desired test sound, or having an intensity within a tolerance of about +/−1.5 dB or less relative to a desired test sound.

Optionally, acceptable fidelity for RCA 100 for the test sound produced by headphones 110 of RCA 100, in accordance with an embodiment of the invention, comprises having a frequency within a tolerance of about +/−4% or less relative to a desired test sound, having an frequency within a tolerance of about +/−2.5% or less relative to a desired test sound, having an frequency within a tolerance of about +/−1.5% or less relative to a desired test sound, or having an frequency within a tolerance of about +/−1% or less relative to a desired test sound.

In a calibration procedure, controller 180 “activates” a digital test signal from DTS database 172 to be converted to a test sound by speakers (not shown) in earpieces 112 of headphones 110. The test sound produced by the headphones is received by microphone 120 and is converted into a digital calibration signal that corresponds to the activated digital test signal. Controller 180 compares the digital calibration signal with the corresponding digital test signal to determine the fidelity of the test sound produced by headphones 110. If the determined fidelity of the test sound is not acceptable for the purpose of testing hearing acuity, controller 180 modifies the digital test signal in DTS database 172 in accordance with the determined fidelity so that the RCA subsequently reproduces the given test sound with acceptable fidelity. Determining fidelity may include determining a difference in intensity or pitch of the produced test sound relative to the desired test sound, or a presence of harmonic distortion in the produced test sound relative to the desired test sound. By way of example, the controller may be configured so that if the intensity of a test sound corresponding to a given digital test signal, as produced by the headphone, is not within a tolerance of +/−3 dB of the desired intensity, the controller modifies the given digital test signal so that the test sound is subsequently produced within +/−3 dB of the desired intensity. By way of another example, the controller may be configured so that if the frequency of a test sound produced by the headphones is not within a tolerance of +/−1.5% of the desired frequency, the controller modifies the corresponding digital test signal so that the test sound is subsequently produced within the tolerance.

In an embodiment of the invention, RCA computer system 200 may comprise a remote system, for example a cloud based computing system, which is schematically represented by a cloud-shaped icon 220. RCA computer system 200 may comprise a computer device 210, which may, by way of example, be a tablet (as shown in FIG. 1), a laptop computer, a desktop computer, or a smartphone. Computer device 210 communicates with cloud computing system 220 through a suitable communications channel schematically represented by a double-sided arrow 230, which may by way of example comprise an internet channel accessed via a wired connection, a Wi-Fi connection, or a cell-phone network. As shown in FIG. 1, headphones 110 and microphone 120 may be operatively connected to cloud computer system 220 via computer device 210.

In accordance with an embodiment of the invention, the RCA comprises a digital-to-analog converter (DAC) 150 that converts the digital test signals from computer system 200 into analog test signals, which is then converted by a speaker (not shown) at earpiece 112 of headphones 110 into the test sounds. RCA 100 may comprise an analog-to-digital converter (ADC) 140 that converts analog calibration signals generated by microphone 120 into digital calibration signals. DAC 150 may optionally be housed in the headphones, in the computer device, for example as a sound card, or, as shown in FIG. 1, in a separate component that connects the computer device and the headphones. ADC 140 may optionally be housed in the microphone, in the computer device for example as a sound card, or, as shown in FIG. 1, in a separate component that connects the computer device and the microphone.

In accordance with an embodiment of the invention, RCA 100 optionally comprises an adapter 130 for acoustically coupling earpieces 112 of headphones 110 to microphone 120. Adapter 130 may comprise an earpiece coupling portion 132 having ends 133 that couple with earpieces 112. Optionally, adapter 130 may be substantially cylindrical in shape. Adapter 130 may further comprise a microphone coupling portion 134, into which microphone 120 may be inserted. For clarity of presentation, headphones 110, microphone 120 and adapter 130 are shown in an exploded view to clearly show each item separately.

Adapter 130 and/or microphone 120 may introduce harmonic distortion and other changes to the test sound produced by headphones 110. The test sound produced by headphones 110 that is registered by microphone 120 and used for the calibration procedure may therefore have differences compared to the same test sound provided to a subject wearing headphones 110. As such, RCA computer system 200 may modify the digital calibration signal to account for the characteristic changes in test sounds introduced by adapter 130 and microphone 120. Microphone 120 may be periodically calibrated so that the modification of the digital calibration signal substantially accurately accounts for the test sound changes introduced by adapter 130 and microphone 120.

DTS database 172 may optionally be stored in cloud computer system 220, as schematically shown in FIG. 1. Optionally, the DTS database may be stored in the computer device 210. Optionally, computer device 210 may download the DTS database from cloud computer system 220 so that the DTS database becomes stored in the computer device. Optionally, cloud computer system 220 may “stream” the activated digital test signal to the headphones for producing the test sound without storing the digital test signal in an intermediate device such as computer device 210. The calibration procedure controlled by controller 180 may optionally be performed in computer device 210, cloud computer system 220, or be distributed in multiple components of RCA computer system 200.

In an embodiment of the invention, DTS database 172 and the calibration procedure controlled by controller 180 are located in cloud computer system 220. A software application (“app”) 182 running in computer device 210 may control transfer of signals from cloud computer system 220 to headphones 110, as well as transfer of signals from microphone 120 to cloud computer system 220 during a calibration procedure.

In an embodiment of the invention, DTS database 172 may be associated with a particular set of headphones 110, which may optionally comprise a particular DAC 150. With each set of headphones having its own headphone-associated DTS database 172, one set of headphones may be used in multiple RCAs 100 without having to repeat a calibration procedure each time the headphone is moved from one RCA 100 to another. Alternatively, a user of RCA 100 may replace one set of headphones with another set without having to do the calibration procedure. The headphone-associated DTS database 172 may be in a memory situated in headphones 110 that provides the headphone-associated DTS database 172 to computer system 200. Alternatively, the memory in the headphones may store an electronic ID (identification) of the headphones readable by computer system 200, and DTS database 172 associated with the electronic ID may be identified by computer system 200 and selected for use. Alternatively, an ID of the headphones could be written on a sticker and be manually provided to computer system 200.

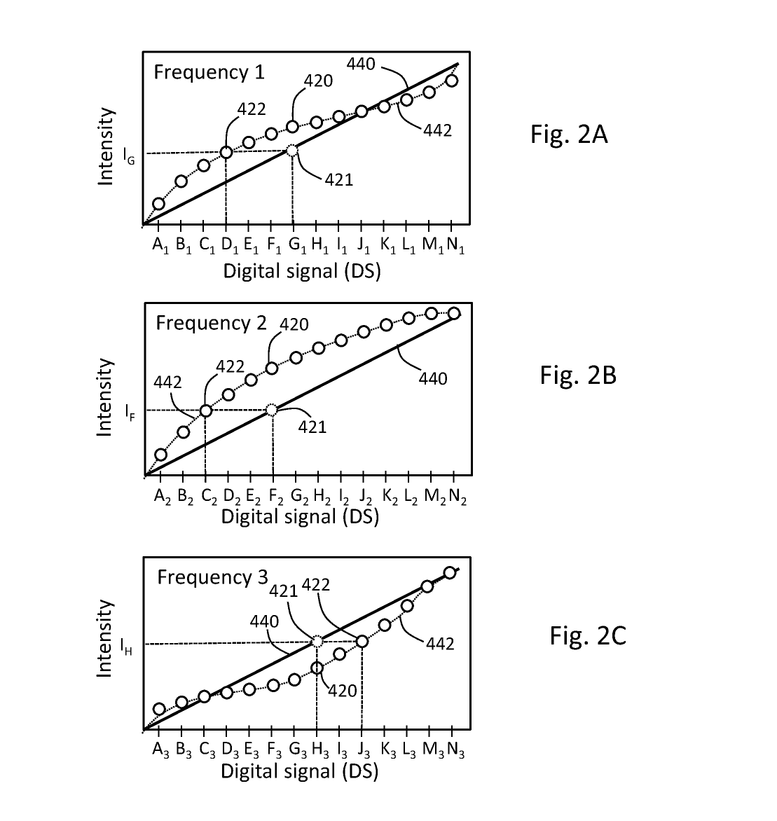

Reference is now made to FIGS. 2A-2C, which show an example of a calibration procedure. In FIG. 2A, a line 440 (“reference line”) for a first frequency (“Frequency 1”) is defined by the desired intensity of a test sound produced by each of digital test signals A₁ through N₁, each of which defines a test sound of a tone having a defined frequency and a defined intensity. Each of signals A₁ through N₁ encodes a tone having a same pitch of frequency 1 and a linearly increasing set of sound intensities. As an example, data plot 421 (shown as a dotted circle) plots the desired intensity (I_{G}) of the test sound produced with signal G₁. During the calibration procedure, the controller activates each of signals A₁ through N₁. For each activated digital test signal, the resulting test sound produced by headphones (for example headphones 110 as schematically shown in FIG. 1) is registered in a microphone (for example microphone 120 as schematically shown in FIG. 2) acoustically coupled to the headphones via an adapter in accordance with an embodiment of the invention. Line 442 (“actual line”) is defined by a plurality of plots 422, which shows intensity of each test sound as registered by the microphone, plotted against the sequence of the corresponding digital test signals. The extent of the discrepancy between actual line 442 and reference line 440 is an indication of the fidelity of the test sounds produced by the RCA. A discrepancy that is sufficiently small indicates acceptable fidelity. For example, the registered intensity of a test sound corresponding to a given signal being within about 3 dB of the signal's desired intensity may be determined by the controller as having acceptable fidelity. The extent of the discrepancy may define the calibration required.

As shown in FIG. 2A, the test sound responsive to digital test signal G₁ is desired to have intensity I_{G}, as schematically represented by plot 421. However, the intensity of the actual test sound produced by the headphones, schematically represented by plot 420, is higher than the desired intensity. The comparison of reference line 440 and actual line 442 shows that the digital test signal with which the headphones would produce a tone at the desired intensity I_{G} is, in fact, D₁, as represented by plot 422. Based on the comparison, the controller updates the calibration dataset so that whenever digital test signal G₁ is instructed to be activated, digital test signal D₁ is activated instead.

As shown in FIGS. 2B and 2C, the discrepancy between the actual line 442 and reference line 440 may be different at different sound frequencies, and the calibration required may be different. At a second sound frequency (“Frequency 2”), as shown in FIG. 2B, the controller updates the DTS database so that, for example, whenever digital test signal F₂ is instructed to be activated, digital test signal C₂ is activated instead. At a third sound frequency (“Frequency 3”), as shown in FIG. 2C, the controller updates the DTS database so that, whenever digital test signal H₃ is instructed to be updated, digital test signal J₃ is activated instead.

Alternatively, the controller may update the DTS database so that a correction factor that modifies intensity, substantially equal and opposite to the measured difference between desired intensity and registered intensity of the test sound, is applied to the digital test signal corresponding to the test sound.

Many calibration protocols are known in the art, and the RCA in accordance with an embodiment of the invention may perform any calibration protocol as known in the art. By way of example, the calibration protocol may be in accordance with OSHA (Occupational Safety and Health Administration), ISO 8253, and ANSI S3.6 standards. By way of example, the OSHA annual audiometer test calls for calibrating the output intensity of tones having one of various test frequencies at 70 dBHL (decibels Hearing Level, which is a dB relative to the quietest sounds that a young healthy individual ought to be able to hear).

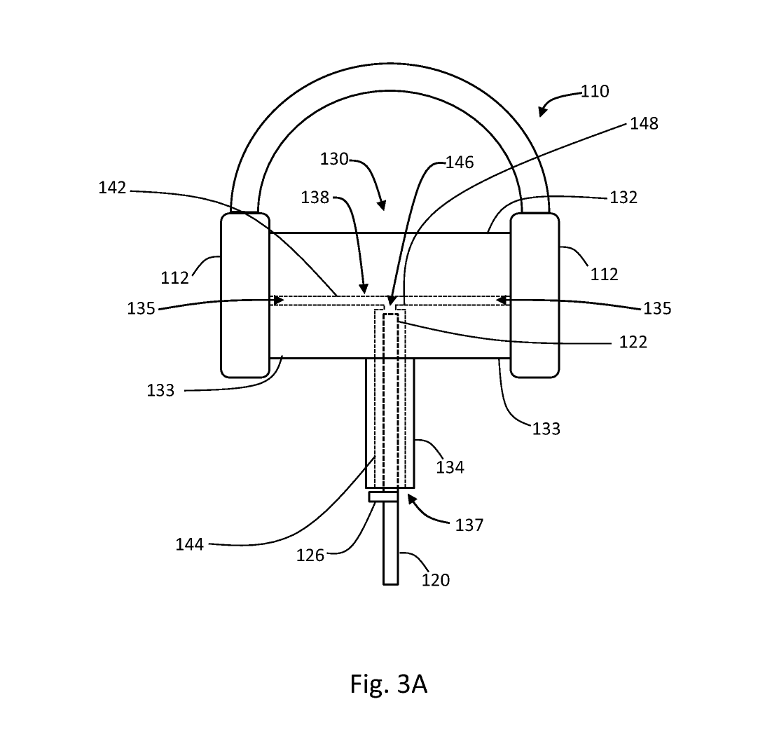

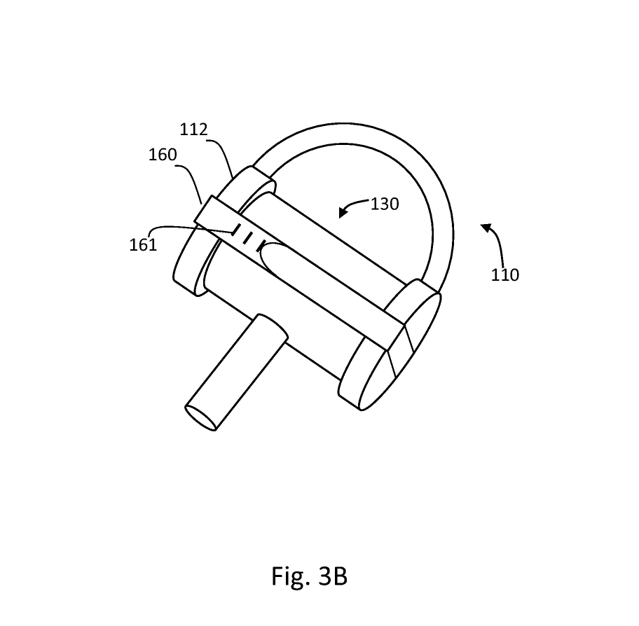

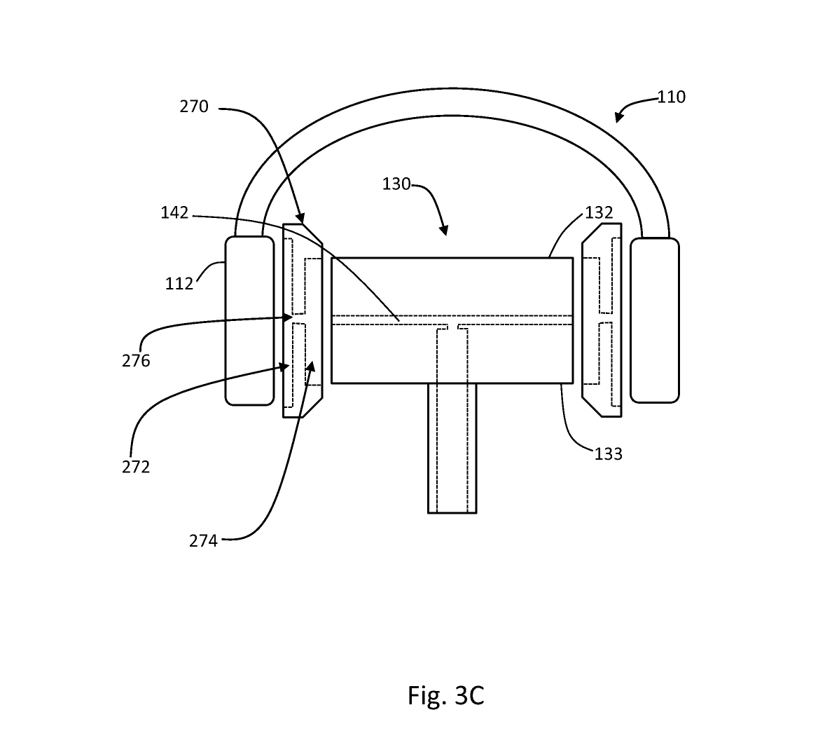

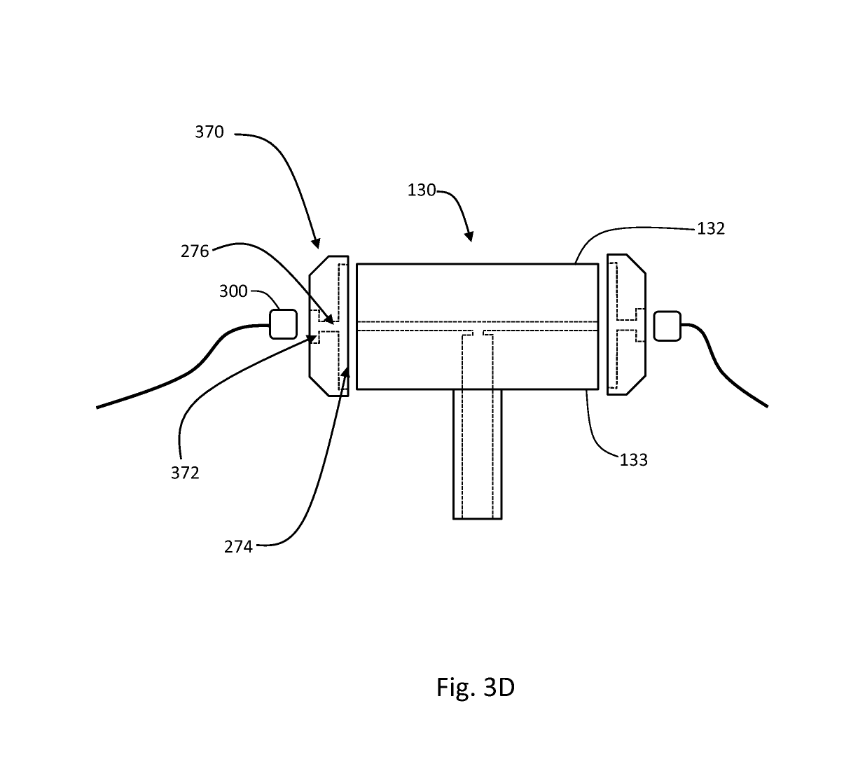

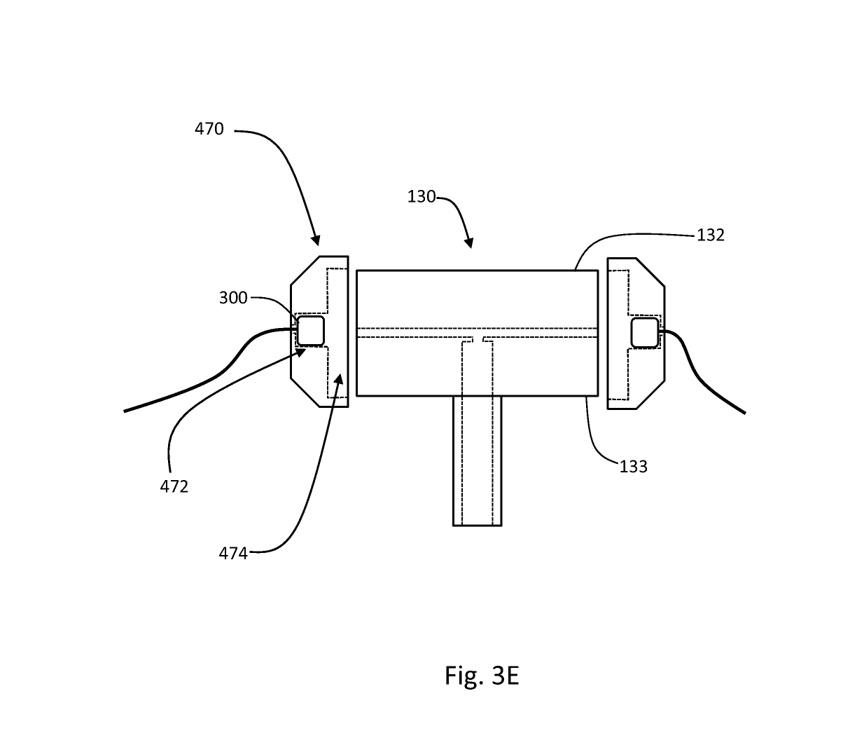

FIG. 3A shows a schematic cross section view of adapter 130 acoustically coupling earpieces 112 of over-ear headphones 110 (by way of example TDH39 headphones) with microphone 120. Ends 133 of earpiece coupling portion 132 securely couple with earpieces 112 in an easily reproducible manner Ends 133 may be rounded or chamfered. Microphone coupling portion 134 may comprise an opening 137 into which microphone 120 may be inserted in an easily reproducible matter. Adapter 130 comprises a T-shaped channel 138, an acoustically isolated sound channel connecting earpieces 112 to microphone 120. T-shaped channel 138 comprises a horizontal channel 142 having openings 135, and a socket 144 having an aperture 146 situated substantially at a midpoint of horizontal channel 142, as well as having opening 137. The cross sectional size of socket 144 may be substantially the same as, or slightly larger than, the cross-sectional size of microphone 120, so that when the microphone is inserted into socket 144, a microphone tip 122 having a sound sensor (not shown) is substantially aligned with aperture 146 and substantially prevented from lateral movement. In an embodiment of the invention, the volume within horizontal channel 142 between aperture 146 and openings 135 is substantially the same on each side.

In an embodiment of the invention, the microphone is near or in contact with aperture 146, so that tip 122 is near or substantially flush with the wall of horizontal channel 142. Socket 144 may comprise a barrier 148 at the interface with horizontal channel 142, which forms an aperture 146 that is optionally narrower than tip 122 of microphone 120. Barrier 148 may serve as a placement guide that allows tip 122 to consistently be placed at a defined position relative to aperture 146 and horizontal channel 142. Alternatively or additionally, microphone 120 may comprise a barrier 126 that serves as a placement guide by setting a predetermined depth to which microphone 120 may be placed inside socket 144. Alternatively or additionally, the diameter of aperture 146 may be substantially the same as the diameter of tip 122. Optionally, aperture 146 and tip 122 may be threaded with matching threads so that tip 122 may be threaded into aperture 146 to securely attach the tip at a predetermined distance near, or flush with, horizontal channel 142.

In an embodiment of the invention, earpieces 112 are secured to respective ends 133 of earpiece coupling portion 132 so that pressure at which each earpiece 112 couples to its respective end is substantially the same. In an embodiment of the invention, earpieces 112 are secured to adapter 130 so that they can be coupled to respective ends 133 with the substantially the same pressure each time the earpieces are coupled to the respective ends. Having earpieces 112 couple to ends 133 with substantially same pressure enhances the calibration process by keeping substantially constant the proportion of the test sounds produced by the speakers in earpieces 112 directed away from microphone 120 through “leakage” of the test sounds out of adapter 130 at or near the interface between earpiece 112 and end 133. By keeping the level of leakage substantially constant between each of said earpieces 112 and/or between multiple securing of earpieces 112 to ends 133, the level of acoustic coupling between earpiece 112 and microphone 120 can be kept substantially constant.

In an embodiment of the invention, earpiece 112 is secured to respective end 133 so that the proportion of the test sound lost through leakage is less than 0.1%, 0.5%, 1%, 2%, 5%, 10% or 15% of the intended intensity of the test sound. Additionally or alternatively, the difference in the intensity of the test sound registered by microphone 120 between the first and the second earpieces is within 0.1%, 0.5%, 1%, 2%, 5%, 10% or 15% of each other, provided that the intended intensity of the test sound from each earpiece 112 is equal. Additionally or alternatively, the difference between the maximum and minimum intensity of the test sound registered by microphone 120 between multiple securings of earpiece 112 to end 133 is about 0.1%, 0.5%, 1%, 2%, 5%, 10% or 15% of the intended intensity of the test sound.

In an embodiment of the invention, earpiece 112 is secured to respective end 133 so that the intensity of the test sound lost through leakage is less than 0.1 dB, is less than 0.2 dB, is less than 0.5 dB, is less than 1 dB, is less than 2 dB, or less than 5 dB. Additionally or alternatively, the difference in the intensity of the test sound registered by microphone 120 between the first and the second earpieces is within about 0.1 dB, about 0.2 dB, about 0.5dB, about 1 dB, about 2 dB, or about 5 dB of each other, provided that the intended intensity of the test sound from each earpiece 112 is equal. Additionally or alternatively, the difference between the maximum and minimum intensity of the test sound registered by microphone 120 between multiple securings of earpiece 112 to end 133 is less than 0.1 dB, is less than 0.2 dB, is less than 0.5 dB, is less than 1 dB, is less than 2 dB, and less than 5 dB.

FIGS. 3B-3C schematically shows headphones 110 having exemplary mechanisms to relatively easily secure the two earpieces to the adapter to achieve a substantially uniform coupling between them.

FIG. 3B schematically shows earpieces 112 of headphones 110 secured onto adapter 130 with a band clamp 160 comprising a band that can securely attach to itself with velcro (not shown) to form a loop. Band clamp 160 may include visual guides 161 so that band clamp 160 may be easily reproducibly formed into a loop having substantially the same perimeter length each time, so that earpieces 112 can be secured to adapter 130 with the substantially the same pressure each time. Alternatively or additionally, the band clamp may comprise a device that directly or indirectly indicates the pressure applied on earpieces 112, by way of example a tension gauge. The band clamp may alternatively comprise a rope (not shown) or band (not shown) that may be secured to itself with, by way of example, a knot (not shown) or a clasp (not shown).

Additionally or alternatively, the earpieces may be secured to the adapter with a clamp, by way of example, a F-clamp (not shown), a C-clamp (not shown), a handscrew (not shown), or a magnetic clamp (not shown). The clamp may include a tension gauge that indicates the pressure applied on the adapter ends by the respective earpieces.

As shown in FIG. 3C, adapter 130 may comprise couplers 270, each coupler 270 having an outer recess 272 and an inner recess 274 connected by a channel 276. Outer recess 272 is dimensioned to snugly hold a given headphone earpiece 112 at a predetermined depth, and inner recess 274 is dimensioned to snugly hold end 133 of earpiece coupling portion 132 at a predetermined depth. Channel 276 may serve to acoustically connect the speaker in earpiece 112 to horizontal channel 142 when headphone 110 and adapter 130 are assembled with coupler 270. Recesses 272 and 274 and channel 276 may be aligned with respect to each other so that, when earpiece 112 is coupled to adapter end 133 with coupler 270, speaker 112 is centered and aligned in an easily reproducible way with respect to horizontal channel 142. Optionally, band clamp 160 as shown in FIG. 3B may be used in combination with couplers 270 to easily and reproducibly couple earpieces 112 to ends 133.

The outer recess of a given coupler may be shaped and dimensioned to be compatible with a particular model of headphone. FIG. 3D schematically shows an alternative coupler 370 that may be substantially the same as coupler 270, having an outer recess 372 dimensioned to snugly hold an earphone insert 300 at a predetermined depth.

FIG. 3E schematically shows an alternative coupler 470 having a first inner recess 472 dimensioned to snugly hold earphone insert 300 and a second inner recess 474 dimensioned to snugly hold an end 133. There is also provided another alternative couple (not shown) that is substantially identical in configuration to coupler 470, in which the first inner recess is dimensioned to snugly hold an earpiece of over-ear headphones (by way of example TDH39 headphones).

By way of numerical example, earpiece coupling portion 132 of adapter 130 has a length of about 14 centimeters (cm). Optionally, earpiece coupling portion 132 has a diameter of about 6 cm and the diameter of horizontal channel 142 is about 0.5 cm. By way of example, the length of microphone coupling portion 134 is about 3 cm and the length of socket 144 is about 5.5 cm. By way of example, the diameter of socket 144 is about 1.25 cm. Optionally, the diameter of aperture 146 is substantially the same as the diameter of horizontal channel 142.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1-25. (canceled)

26. An adapter for coupling sound produced by headphones to a microphone, the headphones comprising first and second earpieces having speakers, the adapter comprising:
a tube having a sound channel that extends between first and second tube ends configured to receive the first and second earpieces respectively; and
a socket having an aperture that communicates with the sound channel to which the microphone may be mounted so that a sound sensor in the microphone is located substantially at the aperture.

27. The adapter according to claim 26, wherein the earpieces are secured to the respective tube ends so that so that a proportion of a test sound produced by each of said earpieces lost through leakage is less than 0.1%, 0.5%, 1%, 2%, 5%, 10% or 15% of the intended intensity of the test sound.

28. The adapter according to claim 26, wherein the earpieces are secured to the respective tube ends so that the difference in intensity as registered by the microphone between a test sound from the first earpiece and the test sound from second earpieces is within 0.1%, 0.5%, 1%, 2%, 5%, 10% or 15% of the intended intensity of the test sound, provided that the intended intensity of the test sound from each earpiece is equal.

29. The adapter according to claim 26, wherein the earpieces are secured to the respective tube ends so that, for each of said first and second earpieces, the difference between a maximum intensity and a minimum intensity of the test sound registered by microphone 120 between multiple securings of the earpieces to the tube end is about 0.1%, 0.5%, 1%, 2%, 5%, 10% or 15% of the intended intensity of the test sound.

30. The adapter according to claim 27, wherein the first and second earpieces are secured to the first and second tube ends, respectively, with at least one clamp.

31. The adapter according to claim 27, wherein the at least one clamp is selected from the group consisting of a F-clamp, a C-clamp, a handscrew and a magnetic clamp.

32. The adapter according to claim 27, wherein the at least one clamp is a band clamp that can form a loop around the first and second earpieces to secure the first and second earpieces onto the first and second tube ends respectively.

33. The adapter according to claim 32, wherein the band clamp comprises visual guides for forming the loop consistently at substantially a same perimeter length.

34. The adapter according to claim 26, wherein the aperture and a tip of the microphone are threaded with matching threads so that the tip can be threaded into the aperture.

35. The adapter according to claim 27, wherein the first and second earpieces are secured to the first and second tube ends, respectively, with a coupler having a first recess dimensioned to snugly seat on one of the tube ends and a second recess dimensioned to snugly receive the respective earpiece.

36. The adapter according to claim 26, wherein the headphones are selected from the group consisting of air-conduction over-ear headphones and earphone inserts.

37. The adapter according to claim 36, wherein the air-conduction over-ear headphones are TDH39 over-ear headphones.

38. An audiometer comprising:
a database storing a non-transitory digital test signal associated with a desired test sound;
a digital to analog converter (DAC) that converts the digital test signal to an analog signal;
headphones that produce a sound responsive to the analog signal;
a microphone that registers the sound;
a controller that adjusts the digital test signal so that the sound produced by the headphones has acceptable fidelity relative to the desired test sound sufficient for the produced sound to be useable in a hearing test; and
an adapter in accordance with claim 26, for coupling sound produced by the headphones to the microphone.
